(19) United States
(12) Patent Application Publication
(10) Pub. No.: US 2016/0320684 A1
(43) Pub. Date: Nov. 3, 2016

(54) METHOD AND APPARATUS FOR CREATION AND ELECTRICAL TUNING OF SPATIALLY NON-UNIFORM REFLECTION OF LIGHT

(71) Applicant: UNIVERSITÉ LAVAL, Québec (CA)

(72) Inventor: Tigran GALSTIAN, Quebec (CA)

(21) Appl. No.: 15/108,880

(22) PCT Filed: Jan. 12, 2015
(86) PCT No.: PCT/CA2015/050016

§ 371 Date: Jun. 29, 2016

Related U.S. Application Data
(60) Provisional application No. 61/926,309, filed on Jan. 11, 2014.

(51) Int. Cl. G02F 1/19; G02F 1/061; G02F 1/1334; G02F 1/1337; G02F 1/133; G02F 1/1343; G02F 1/1335; G02C 7/08; G02C 7/12; G02F 1/13363; G02C 7/04; G02F 1/29; H04N 5/232; A61F 2/16; G02F 1/137

(52) U.S. Cl. CPC G02F 1/19; G02F 1/137; G02F 1/061; G02F 1/1334; G02F 1/1337; G02F 1/13306; G02F 1/134327; G02F 1/133553; G02C 7/083; G02C 7/12; G02F 1/13363; G02C 7/04; G02F 1/29; H04N 5/23296; H04N 5/23287; A61F 2/1627; G02F 2001/13775; G02F 2203/18; G02F 2001/294

(57) ABSTRACT

A variable optical device for controlling properties of reflected light is described. The device includes a light reflecting surface, a layer of dynamically controllable material and an excitation source for generating an excitation field acting on the layer of dynamically controllable material. An electrical drive signal applied to the excitation source causes a change of optical properties in the layer of dynamically controllable material to provide a spatially varying change in light reflection having at least one of a desired phase curvature and a desired amplitude modulation profile.

### RELATED APPLICATIONS

This application is a regular filing of, and claims priority from, U.S. Provisional Patent Application 61/926,309 of the same title filed Jan. 11, 2014, which is incorporated herein by reference.

### TECHNICAL FIELD

The present invention relates to the field of electrically tunable optical reflective devices. More particularly, the proposed solution is directed to a method and apparatus for creating, and electrically tuning, a spatially non-uniform reflection of light using liquid crystal materials.

### BACKGROUND

In many photonic applications, control of the divergence of light beams is required. As is known, light properties may be changed in both transmission mode and reflection mode, the latter being particularly important in applications such as tunable laser cavities, stabilized holographic systems, etc.

Legacy solutions are mainly based on mechanical movement of the position of a mirror (for example, using a piezo element) or on mechanical variation of the curvature of the mirror (bending, torsion, etc.)

FIG. 1A illustrates a prior art fixed curved mirror which provides light reflection generating a fixed phase curvature. As shown, an incident light beam 2 having a flat incident phase plane 3 and incidence angle 6, measured with respect to mirror normal 5, is reflected by curved mirror 1. The reflected beam 4 is characterized by a curved reflected phase plane 7. In this case, the position of the mirror 1 must be changed to change the optical parameters of the overall system. Unfortunately, this mechanical movement may be problematic to the overall functionality of the system (for example in terms of vibration, motion settling time, backlash, etc.)

As another example, FIGS. 1Ba) to 1Be) illustrate various prior art configurations of laser cavities having fixed curvature reflectors (two mirrors with curvature radiuses R_{1,2} placed a distance L apart), where the mirror curvatures and their reflectivity profiles are fixed and important to operate a laser in stable or unstable modes. In this case, the movement of a mirror may cause the cavity to be tuned in or out of a corresponding stability zone. Illustrated stability zones include a) plane-parallel, b) concentric (spherical), c) confocal, d) hemispherical and e) concave-convex.

Several approaches have been explored whereby, instead of mechanically moving a mirror, the curvature of the mirror is changed. One prior art solution is the use of multiple Micro-Electro-Mechanical-Systems (MEMS) elements 111 spread over a surface of a reflecting device 1, providing variable surface curvature light reflection as shown in FIG. 1C. Unfortunately, this solution is costly, vulnerable, and fails to provide a spatially continuous operation and control; providing instead a pixilated operation and control. This solution is also based on mechanical movement, which is less than ideal.

Other mechanical solutions have also been proposed, such as the use of a deformable membrane, for example.

However, motion-less (or motion-free) solutions have advantages making them more appealing. Motion-free electrically controlled (dynamically variable) uniform reflection is known and largely used in Liquid Crystal Display (LCD) technologies such as described by L. M. Blinov, V. G. Chigrinov in “Electro-optic effects in Liquid Crystal Materials”, Springer-Verlag, N.Y., 459 pp, 1994. FIG. 2 shows an example of a prior art tunable reflective LCD. Each reflective LCD pixel (or unit) includes a layer of dynamically controllable material 8 which is uniform along an x axis (for example, liquid crystal or polymer composite), as well a fixed mirror 9 of high reflectivity which is also uniform along axis x. One important differentiating aspect of such a dynamically variable mirror is the uniform character of reflection of each LCD pixel. That is, the wavefront curvature (or intensity profile) of the reflected light from each pixel is not modulated across the given pixel. Modulation may only be achieved over the greater LCD panel using different voltages applied to multiple pixels, which again introduces spatially discontinuous operation (granularity problems), is costly to manufacture and increases control complexity (for example separate control for each pixel).

Another prior art solution uses multiple (more than 2) transparent electrodes such as Indium Tin Oxide (ITO) distributed on a Liquid Crystal (LC) cell substrate as described by S. T. Kowel, P. G. Kornreich, D. S. Cleverly in “Adaptive liquid crystal lens”, U.S. Pat. No. 4,572,616, 1986 (filed August 1982) and by N. A. Riza, M. C. DeJule in “Three-terminal adaptive nematic liquid-crystal lens device”, Opt. Lett. 19, pp. 1013-1015, 1994. Although motion-free, this solution is still limited because of its granularity (spatially discontinuous operation) and control complexity (separate drive for each one of the multiple electrodes). The use of two or less (instead of multiple) electrodes could significantly reduce the cost and complexity of the device.

Unfortunately, all of these prior art solutions have performance and/or manufacturing problems, due in part to the fact that the solutions were originally designed for operation in transmission mode only.

### SUMMARY

In contrast with the prior art solutions, the proposed solution provides a method and apparatus for electrically controlling a variable optical reflective device using non-uniform excitation instead of using multiple pixel separately controlled elements. In a specific example, a spatially non-uniform excitation field, which can be for example an electric field or a magnetic field, is generated by two electrodes and is used to control the optical properties such as index of refraction or absorption of a layer of dynamically controllable material, such as a nematic liquid crystal layer, within the optical reflective device.

The proposed solution also provides a method and apparatus for electrically controlling a variable optical reflective device using non-pixellated planar (standard) liquid crystal cells or composite polymer films, for example located on a surface of a total internal reflection element.

The use of such an electrically controlled variable optical reflective device to generate electro-optical tuning/control of reflection phase and amplitude with low losses and a simpler construction and/or manufacture is also described herein.

In accordance with an aspect of the proposed solution there is provided a variable optical device for controlling properties of reflected light, the device comprising: a light reflecting structure; a layer of continuous non-pixelated dynamically controllable material; and an excitation source for generating an excitation field acting on said layer of dynamically controllable material, wherein an electrical drive signal applied to said excitation source causes a change of optical properties in said layer of dynamically controllable material to provide a spatially varying change in light reflection having at least one of a desired phase curvature and a desired amplitude modulation profile.

In accordance with another aspect of the proposed solution there is provided a device wherein said layer of dynamically controllable material is sandwiched between a pair of alignment layers and comprises nematic liquid crystal material.

In accordance with further aspect of the proposed solution there is provided a device wherein each of said pair of alignment layers has an alignment direction, said pair of alignment layers being oriented in one of same direction and opposing direction with respect to each other.

In accordance with further aspect of the proposed solution there is provided a device wherein said excitation field generated by said electrode system is spatially non-uniform, wherein said spatial non-uniform electrode system is configured to generate a spatially non-uniform field obtained by lateral attenuation of a potential across a combination of electrode system geometry and by electrical and optical properties of adjacent materials without using individual control of a plurality of pixels.

In accordance with further aspect of the proposed solution there is provided a device wherein said electrode system has a first group of electrodes which is non-uniform or segmented and said second electrode group is uniform, wherein said first group of non-uniform electrodes includes a hole patterned electrode and a weakly electrically conductive layer.

In accordance with further aspect of the proposed solution there is provided a device wherein said dynamically controllable material is liquid crystal mixture or polymer composite that is sensitive to the said excitation field.

In accordance with further aspect of the proposed solution there is provided a device wherein said liquid crystal mixture or polymer composite comprises polymer stabilized nematic liquid crystal layer.

In accordance with further aspect of the proposed solution there is provided a device wherein said layer of liquid crystal mixture is characterized by one of: a spatially non-uniform liquid crystal cell alignment and a spatially uniform liquid crystal cell alignment.

In accordance with further aspect of the proposed solution there is provided a device wherein said light reflecting structure is one of a metal mirror, a dielectric mirror, a plurality of dielectric layers and a total internal reflection interface.

In accordance with further aspect of the proposed solution there is provided a tunable optical device for controlling the properties of reflected light, said device having a variable light reflection phase curvature, controlled essentially by an electrical drive signal.

In accordance with further aspect of the proposed solution there is provided a tunable optical device further comprising an active polarization rotator configured to select between two polarizations of light.

In accordance with further aspect of the proposed solution there is provided a tunable optical device for controlling the properties of reflected light, said device having a variable light reflection amplitude spatial distribution, controlled essentially by an electrical drive signal.

In accordance with further aspect of the proposed solution there is provided a tunable optical device in combination with additional optics to form incident and reflected beams in counter propagation, co-propagation and angled (e.g. cross) propagation geometries.

In accordance with further aspect of the proposed solution there is provided a combination of at least two controllable non-uniformly reflective devices and additional optics including an image sensor to form one of an optical zoom system, an autofocus system and image stabilization system in a mobile camera.

In accordance with further aspect of the proposed solution there is provided an array of controllable non-uniformly reflective devices in combination with additional optics, such as an origami kind lens, to form one of an optical zoom system and an autofocus system, wherein said array is one of: periodic, aperiodic, concentric and linear.

In accordance with further aspect of the proposed solution there is provided a contact lens or an intraocular lens for enhancing vision, the lens comprising: an array of controllable non-uniformly reflective devices in combination with additional optics, such as an origami kind lens; a first integrated polarizer layer having a first polarizing orientation over a central area of said origami lens; a second integrated polarizer layer having a second polarizing orientation over a peripheral area of said origami lens; and an integrated polarization rotator layer in a combined optical path of incident light passing through said first and second polarizer, said polarization rotator being configured to select between central area vision and peripheral vision for selecting between normal and zoomed vision.

In accordance with further aspect of the proposed solution there is provided a lens, wherein at least one of said non-uniform reflective devices comprising a group of segmented electrodes in a transversal plane configured to steer reflected light inside an eye to change an imaging area on a retina of said eye.

In accordance with further aspect of the proposed solution there is provided a variable optical device wherein one of a phase and an amplitude of reflected light is controlled using two liquid crystal material layers arranged in cross oriented layers so as to provide polarization independent operation.

In accordance with further aspect of the proposed solution there is provided a variable optical device wherein a phase or amplitude of reflected light is controlled using liquid crystal material arranged in a single layer in combination with a birefringent plate so as to provide polarization independent operation.

In accordance with further aspect of the proposed solution there is provided an array of controllable non-uniformly reflective devices in combination with at least one photovoltaic cell configured to steer solar incident light to compensate for solar movement.

In accordance with yet another aspect of the proposed solution there is provided an array of controllable non-uniformly reflective devices further configured to focus said solar incident light onto said photovoltaic cell.

### BRIEF DESCRIPTION OF THE DRAWINGS

The invention can be better understood by way of the following detailed description of embodiments of the proposed solution with reference to the appended drawings, in which:

FIG. 1A is a schematic representation of a prior art curved fixed mirror and its reflection properties;

FIG. 1Ba) to 1Be) illustrate various prior art laser cavity configurations and associated laser cavity stability regions;

FIG. 1C is a schematic representation of a prior art curved tunable mirror using MEMS elements and its reflection properties;

FIG. 2 is a schematic representation of a prior art configuration of a dynamically variable uniform mirror used in traditional reflective LCDs using individual control for each of multiple individual pixels;

FIG. 3A is a schematic representation (in cross-section) of a dynamically variable and spatially non-uniform mirror configuration using an initially uniform controllable material and non-uniform excitation source according to a non-limiting implementation of the proposed solution;

FIG. 3B illustrates spatially variable excitation of the refractive index of the dynamically controllable material for the mirror illustrated in FIG. 3A;

FIG. 3C illustrates spatially variable excitation of the light phase modulation for the mirror illustrated in FIG. 3A;

FIG. 3D is a schematic representation of a dynamically variable and spatially non-uniform mirror configuration using a uniform excitation source and non-uniform controllable material according to a non-limiting implementation of the proposed solution;

FIGS. 4A and 4B schematically respectively represent a geometry and principle of operation of a dynamically variable non-uniform liquid crystal mirror according to a non-limiting implementation of the proposed solution;

FIGS. 5A to 5G are schematic non-limiting examples of non-uniform “back” electrode configurations for the liquid crystal mirror illustrated in FIG. 4A;

FIG. 6 is a schematic representation of a polarization-independent mirror using two cross-oriented Liquid Crystal Layers (LCLs) according to a non-limiting implementation of the proposed solution;

FIG. 7 is a schematic representation of a polarization-independent mirror using one LCL with a quarter-wave retardation layer according to a non-limiting implementation of the proposed solution;

FIGS. 8A and 8B schematically represent a high transparency and high optical power resistant tunable liquid crystal mirror, according to a non-limiting implementation of the proposed solution;

FIG. 8C schematically illustrates, in frontal view, the geometry of a segmented ring electrode in accordance with the proposed solution;

FIG. 9 is a schematic representation of a polarization independent LC mirror using one common floating conductive layer correcting the light wavefront according to a non-limiting implementation of the proposed solution;

FIG. 10A is schematic representation of a polarization dependent LC mirror using multiple transparent concentric ring electrodes 12 some of which are coupled by resistive bridges and others are connected to power supplies according to a non-limiting implementation of the proposed solution;

FIG. 10B is a plan view of the electrode structure 12 of FIG. 10A in accordance with the proposed solution;

FIG. 10C is a plan view of a segmented electrode structure 12 for use in controlling aberrations for a LC tunable mirror geometry as illustrated in FIGS. 10A and 10B in accordance with the proposed solution;

FIG. 11A is a schematic diagram illustrating another embodiment of the proposed solution;

FIG. 11B is a schematic diagram illustrating a further embodiment of the proposed solution;

FIGS. 12A and 12B schematically illustrate a top view and a cross-sectional view, respectively, of a bipolar liquid crystal tunable mirror geometry in accordance with another embodiment of the proposed solution;

FIG. 13 schematically illustrates another bipolar liquid crystal tunable mirror geometry in accordance with another embodiment of the proposed solution;

FIG. 14A is a schematic representation of a configuration for an angle reflecting tunable mirror, allowing a double passage of reflected light through the controllable material, according to a non-limiting implementation of the proposed solution;

FIG. 14B is a schematic representation of a configuration for an angle reflecting tunable mirror, allowing a variable evanescent (partial) or complete penetration of reflected light into the controllable material, according to a non-limiting implementation of the proposed solution;

FIG. 15A to 15C schematically illustrate three variant assemblies of the angle reflecting tunable mirror, according to non-limiting implementations of the proposed solution;

FIGS. 16A to 16E schematically illustrate different applications of a tunable reflective mirror, according to non-limiting implementations of the proposed solution;

FIG. 16F schematically illustrates a three dimentional perspective view of an optical system providing both optical zooming and image stabilization using tunable reflective mirrors, according to a non-limiting implementation of the proposed solution;

FIG. 16G schematically illustrates an optical system providing optical steering and focus of a light source (e.g. LED, laser, etc.) using a tunable reflective mirror, according to a non-limiting implementation of the proposed solution;

FIG. 16H schematically illustrates another optical system providing optical steering and focus of incident light (e.g. from the sun, etc.) using a tunable reflective mirror, according to another non-limiting implementation of the proposed solution;

FIG. 17 schematically illustrates the use of tunable reflective elements to build a dynamically variable non-uniform pinhole, according to a non-limiting implementation of the proposed solution;

FIG. 18A schematically illustrates, in partial cut-out, a prior art of “Origami”-type lens and FIG. 18B, the cross-section of, one embodiment of the proposed solution in which one or more tunable reflective elements are used to build an electrically variable flat imaging telephoto lens with auto-focus or optical zoom properties;

FIGS. 19a) to 19c) schematically illustrate a prior art contact lens configured to selectively provide low light augmented vision and/or telescopic function;

FIG. 20 schematically illustrates an integrated tunable contact lens configured to enhance central vision in accordance with an embodiment of the proposed solution;

FIG. 21 schematically illustrates a tunable contact lens configured to redirect incident light onto usable portions of the retina in accordance with the proposed solution;

FIG. 22 schematically illustrates a tunable contact lens configured to provide zooming functionality in accordance with the proposed solution; and

FIG. 23 schematically illustrates an integrated tunable contact lens configured to switch between central vision and peripheral vision in accordance with the proposed solution,

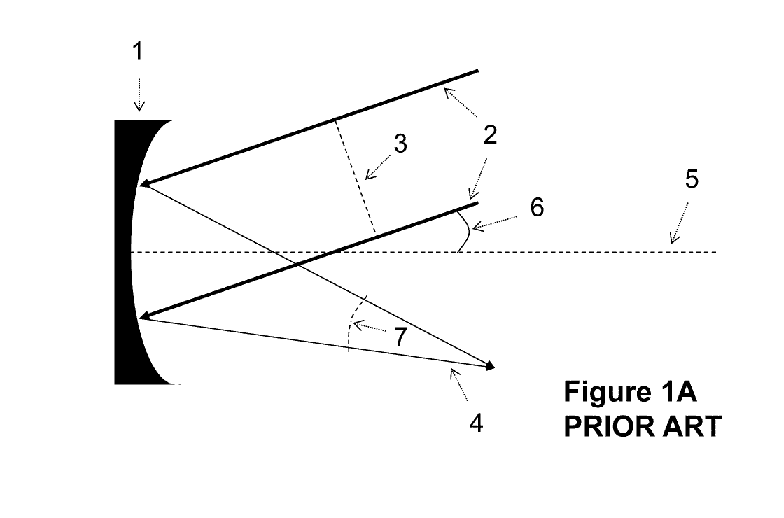

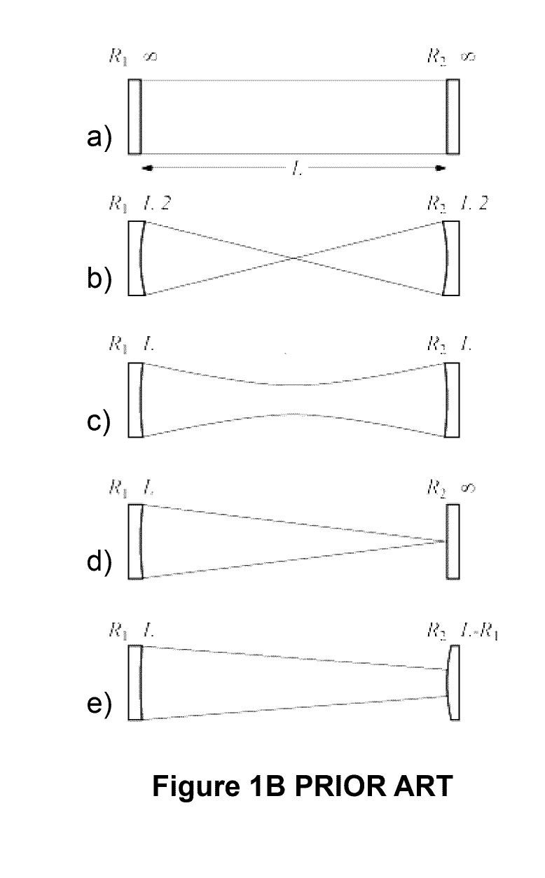

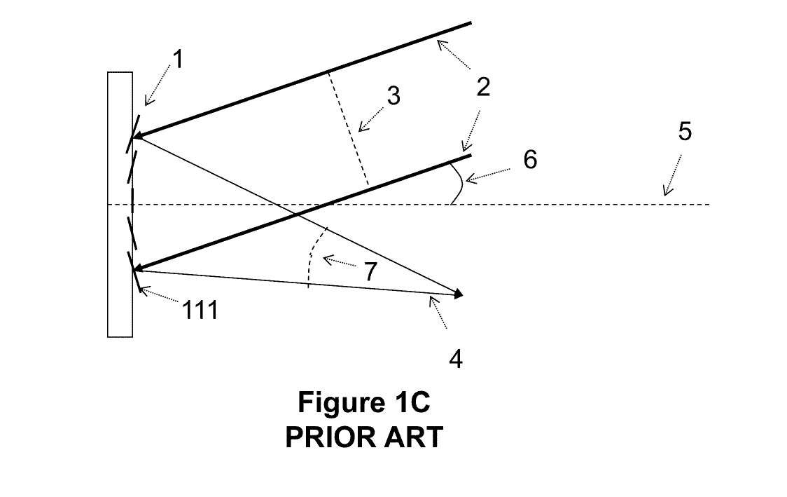

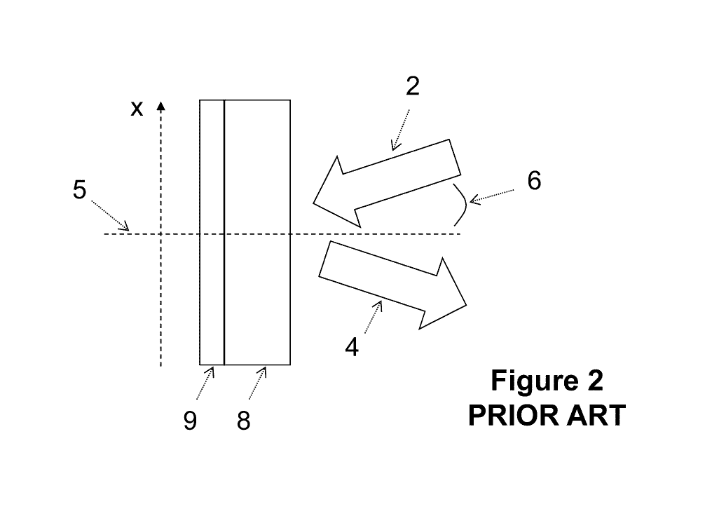

wherein same labels refer to similar features throughout the figures.

### DETAILED DESCRIPTION

The proposed solution is directed to reducing light flux loss and reduced cost of a variable optical reflective spatially continuous (non-pixellated) device which is electrically controllable using either a spatially non-uniform excitation field (electric, magnetic, thermal, acoustic, etc.) or non-uniform controllable material layer, such as liquid crystal cells or composite polymers. Such a device can be used for tunable reflection, diffracting, steering, etc.

In contrast with the above-discussed prior art solutions, which have been designed for operation in transmission mode only, reflection mode electrically controllable devices are described in accordance with the proposed solution. Employing a reflection geometry allows the use of a much broader range of: excitation methods; electrodes (including optically non-transparent); electro-magnetic, acoustic or thermal excitation sources, at least some of which improve control ability and significantly facilitate manufacture thereof while reducing cost. Improved performance and manufacturing advantages with respect to the known prior art electrically controllable reflection devices are achieved. For example, in some implementations described herein, the light path does not traverse electrode layers, which improves both the transmission (output) and high-power resistance (reliability) of such a device. As another example, by placing a control electrode structure behind the reflective surface of the tunable mirror, a greater choice of electrodes, electrode forms, and electrode material compositions are available which can reduce manufacturing constraints.

For the sake of simplicity, the following description outlines refractive structures, while other types of structures (e.g., diffractive) and more complex combinations of elements can equally be used. Similarly, embodiments using static or electro-optic materials are described, it being understood that other materials can be used instead to obtain the same goal, which is reduced cost, reduced loss, and improved efficiency of operation.

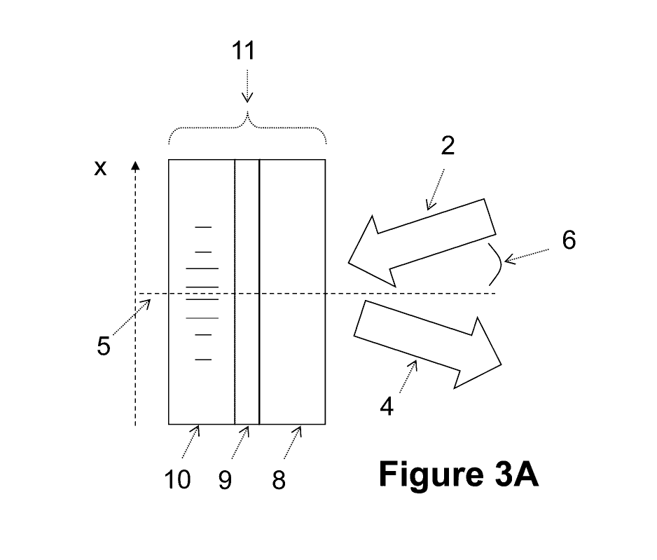

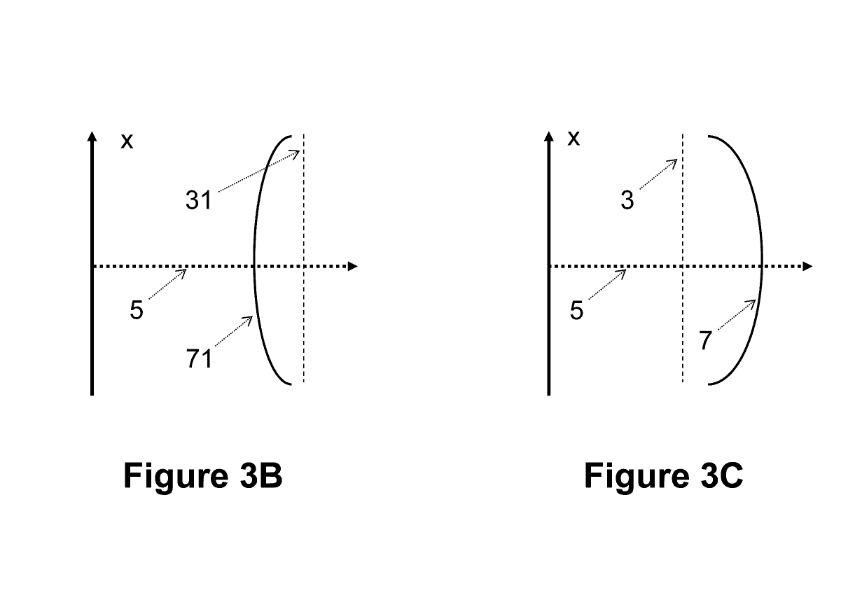

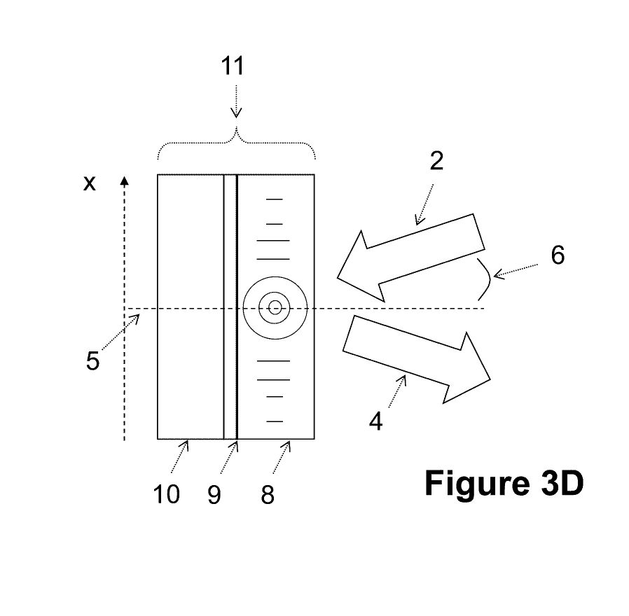

FIG. 3A schematically illustrates a dynamically variable non-uniform mirror geometry (configuration), according to a non-limiting embodiment of the proposed solution. The mirror 11 geometry includes a layer of dynamically controllable uniform material 8 such as, for example, a uniform Liquid Crystal (LC) layer, a reflecting surface 9 (e.g. fixed mirror, total internal reflection prism, dielectric multi-layer structure, etc.) and a source of a spatially non-uniform excitation field 10 (e.g. electric, magnetic, thermal, acoustic, etc.) which is spatially variable along an x axis and is dynamically variable. For example, using this configuration it is possible to generate a gradient of reflection employing two electrodes generating an electric field therebetween. For example, a gradient of refraction can be provided by employing a LC layer containing nematic LC. One of the two electrodes, the excitation source 10, is notably at least partially hidden behind the reflecting surface 9 (which is at least partially transparent for the excitation field) for example generating a non-uniform (linear, circular or other type of gradient-like) excitation of the dynamically controllable uniform material 8, e.g. the (nematic) LC layer. A single second electrode can be used in front of the reflecting surface (not shown), but not necessarily in the optical path, to provide the above mentioned excitation. The mirror 11 is tunable as is the phase curvature of its light reflection 4 (and in some cases, the amplitude of the reflected light also).

FIGS. 3B and 3C schematically illustrate principles of operation of a LC mirror for curved phase generation via spatially variable dynamic excitation in accordance with a non-limiting implementation of the proposed solution. FIG. 3B illustrates an induced spatial variation (along x axis) of refractive index n of the controllable material (8) due to excitation provided by the excitation source (10). The refractive index distribution 31 of the dynamically controllable uniform material (8) before excitation is illustrated by dashed lines, while the refractive index distribution 71 of material (8) during the excitation is illustrated in solid line. FIG. 3C illustrates the phase profile 3 of the incident beam and the light phase modulation 7 (Δφ) of the reflected beam 4 during excitation.

Alternatively, a gradient of reflection is generated with a variant mirror configuration in which the excitation source generates a uniform excitation of a dynamically controllable material 8, which is spatially non-uniform (lens-like), as illustrated in FIG. 3D. In accordance with a specific non-limiting example, the application of a uniform electric or magnetic field to a spatially non-uniform LC layer using one reflective surface 9 provides a mirror 11 having a performance similar that illustrated in FIG. 3A in which the excitation source 10 is spatially variable, wherein the nematic LC layer alignment is gradually changed. Examples of spatially non-uniform dynamically controllable materials are described in U.S. Pat. No. 7,218,375, U.S. Pat. No. 7,667,818, U.S. Pat. No. 8,031,323 all claiming priority from U.S. Provisional Patent Application 60/475,900 filed 2003 Jun. 5, all of which are incorporated herein by reference. One non-limiting example of spatially non-uniform dynamically controllable material includes (a layer of) polymer stabilized nematic liquid crystal in a polymer matrix.

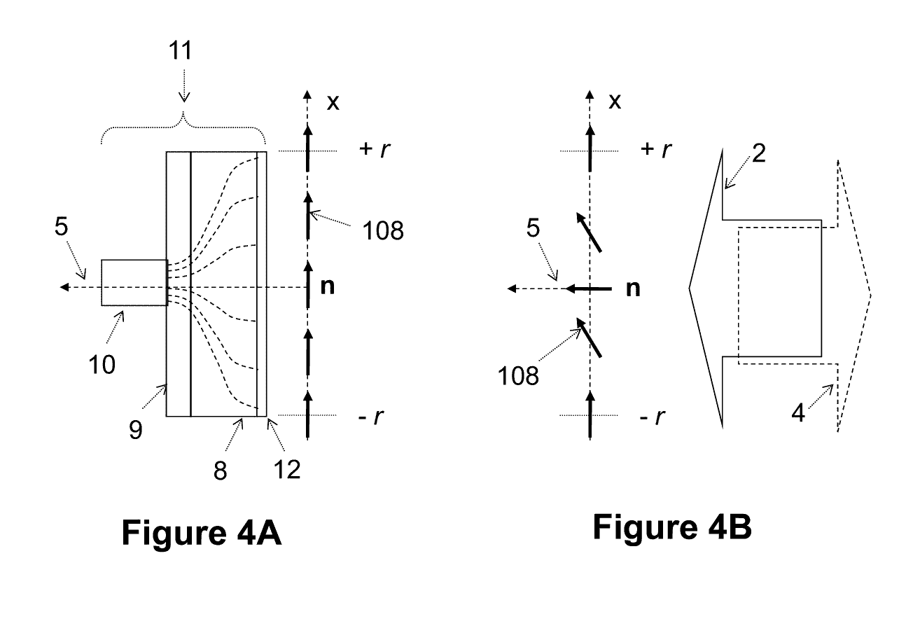

FIGS. 4A and 4B schematically illustrate a LC mirror and the principle of operation of a dynamically variable non-uniform LC mirror characterized by curved phase generation via non-uniform excitation, according to a non-limiting implementation of the proposed solution. In this example, the tunable mirror 11 geometry (configuration) includes a pair of electrodes, notably a back electrode 10 which is spatially non-uniform (for example having limited extent in the x direction over the active working area of the mirror 11) as well a front electrode 12 which is uniform and optically transparent. A schematic representation of the electric field is illustrated as electric field lines 13. An optical mirror 9 is transparent (at least partially) to the excitation field (13) action. Director vectors n illustrate the average ±orientation of nematic LC long molecular axes, and r is the radius of the mirror. As illustrated in FIG. 4B, the phase retardation profile of the reflected beam 4 can be spatially modulated by spatially modulating the orientation of the directors n, which are attracted to and/or repulsed by the electric field lines.

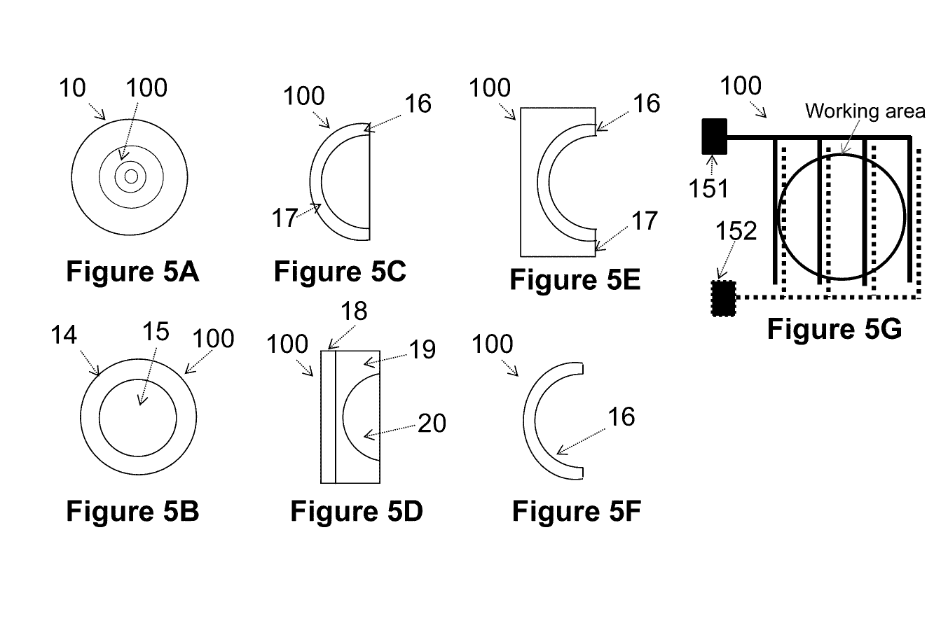

FIGS. 5A to 5G illustrate non-limiting examples of back electrodes which can provide non-uniform excitation for the LC mirror configuration illustrated in FIGS. 4A and 4B. FIG. 5A illustrates a front view (in the plane parallel to the plane of the controllable material) of an electrode 100 including a spatially varying resistive electrode. FIG. 5B illustrates a front view of a ring-shaped 14 electrode 100 or a localized 15 electrode (for example a point electrode as illustrated in FIG. 4A). FIG. 5C illustrates a side view of electrode 100 as a concave electrode 16 on a curved surface 17. In FIG. 5D, the electrode 100, illustrated in side view, has a planar electrode 18 combined with spatially non-uniform dielectric layer or semiconductors 19 and 20. In FIG. 5E (side view), the back electrode 100 includes a concave electrode 16 on a concave curved surface 17. In FIG. 5F (side view), the back electrode 100 is a curved electrode 16 for example a concave mirror. In FIG. 5G (front view), the back electrode 100 is a combination of pairs of linear interdigitating electrodes 151 and 152.

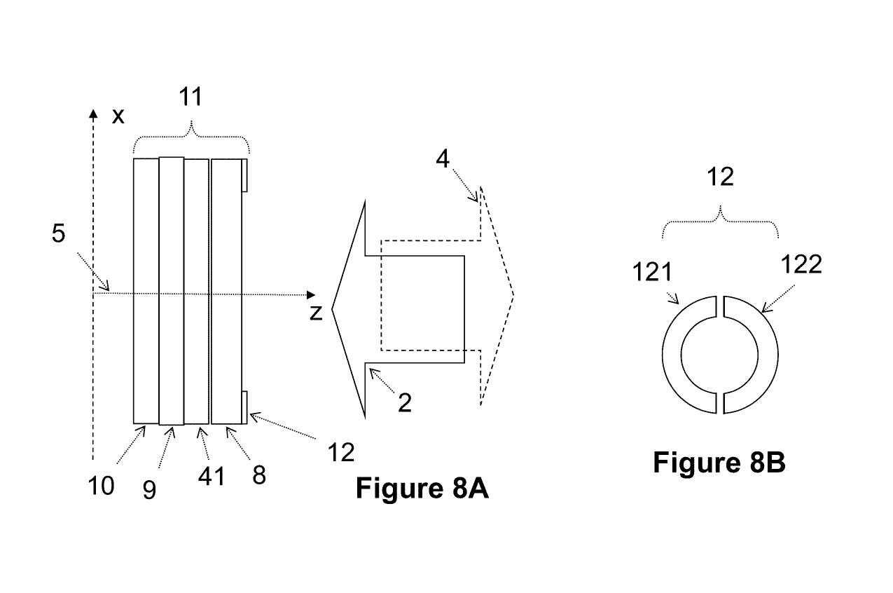

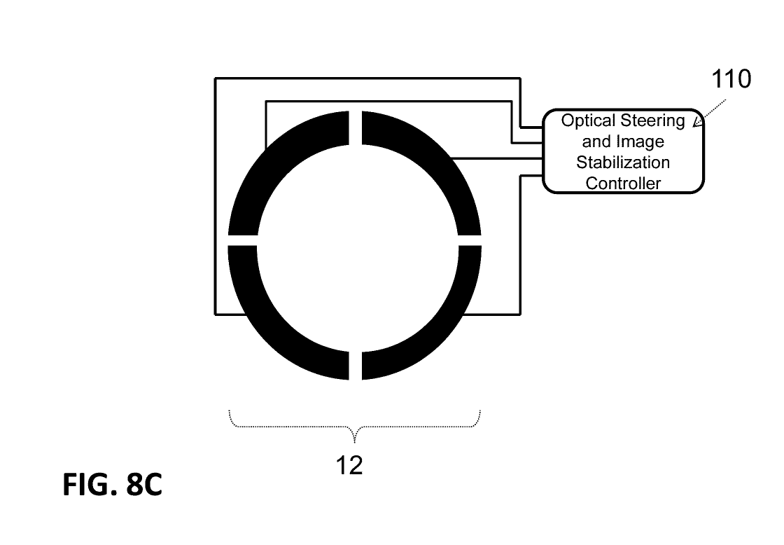

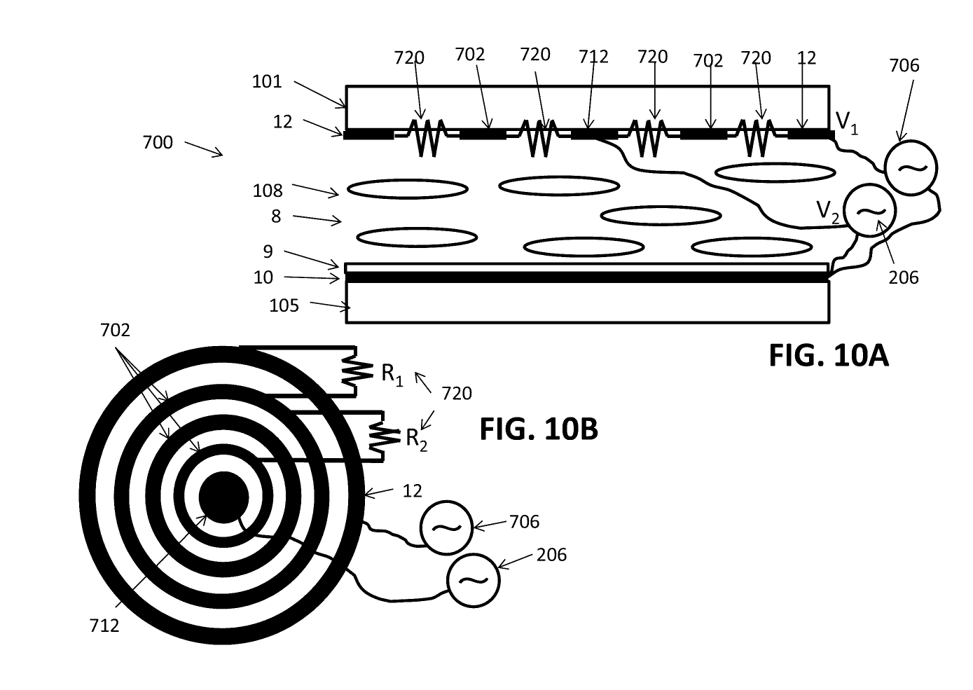

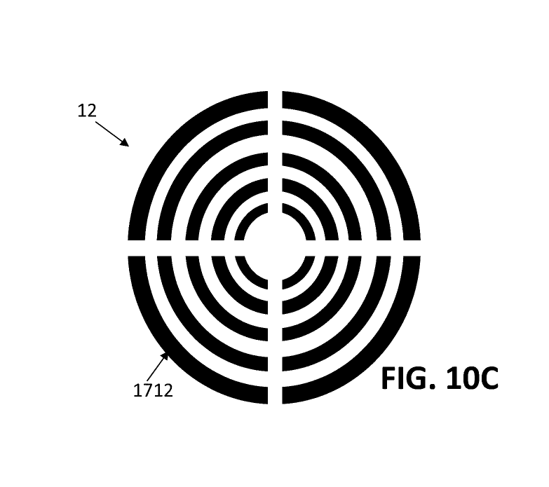

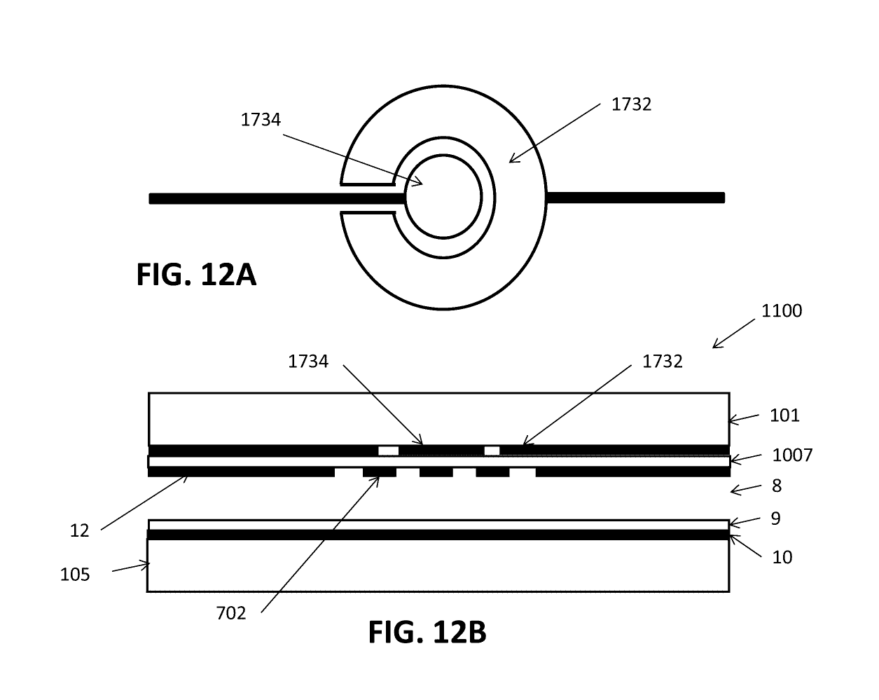

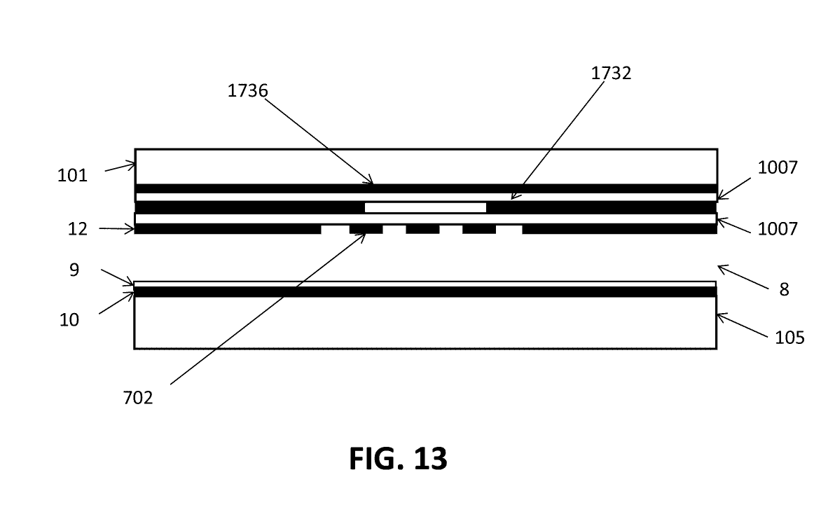

For clarity, many other types of excitation sources 10, including some dynamically variable in form or in function, can be used in the above mentioned application. For certainty, the invention is not limited to these examples, other types of electrodes can be used including segmented electrodes (FIG. 8B, FIG. 8C, FIG. 10C), juxtaposed electrodes, coupled electrodes (FIG. 12A, FIG. 12B, FIG. 13), etc.

It should be noted that, within the LC mirror 11, the reflector 9 can be removed and the excitation source (the “electrode” 100) itself can play the role of the reflector of light (for example, the elements 10, 15, 16, 18 of FIGS. 5A to 5G).

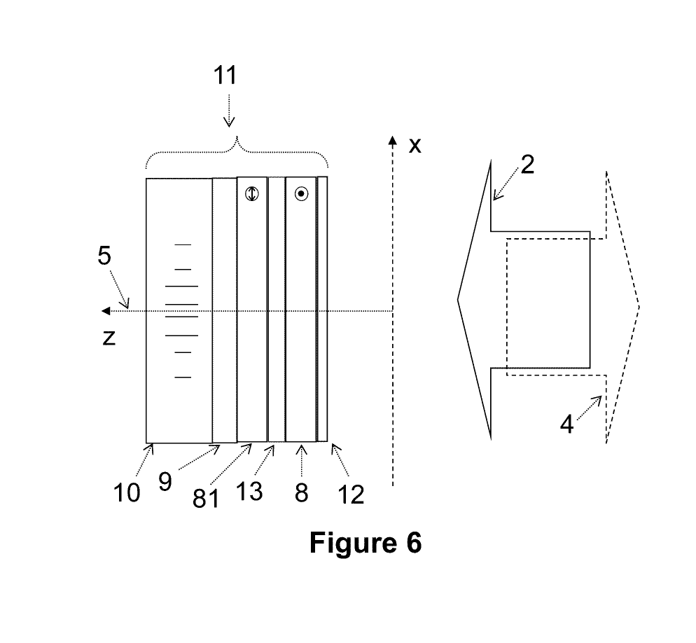

FIG. 6 is a schematic representation of a polarization independent double-layer LC mirror according to a non-limiting implementation of the proposed solution. In this case, the use of two crossed (directors laying in perpendicular planes with respect to one another, and each perpendicular to the normal 5) LC layers 8 and 81 within the optical reflective device 11, separated by spacer substrate 130, makes the device polarization independent. The LC layer 81 has a director orientation which is perpendicular to the director orientation of LC layer 8.

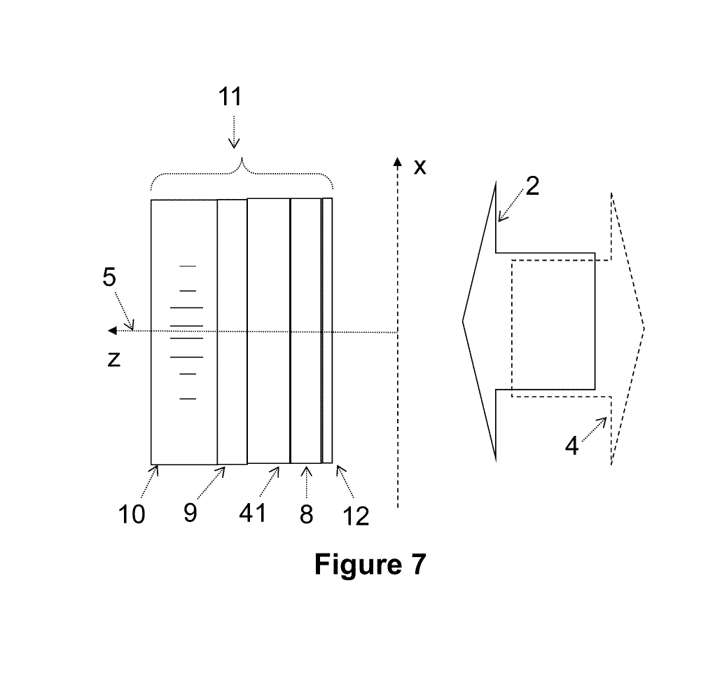

FIG. 7 is a schematic representation of a polarization independent single layer liquid crystal mirror 11 according to a non-limiting implementation of the proposed solution. In this case, a broad band quarter wave retardation layer 41 is employed between single liquid crystal layer 8 and the mirror layer 9. Regardless of an alignment direction of the liquid crystal molecular directors in layer 8, an incident light beam 2 is divided into an ordinary polarized incident light beam which passes unaffected through the liquid crystal layer 8 towards the broad band quarter wave retardation layer 41, and into an extraordinary polarized incident light beam which is orthogonal to the ordinary polarized incident light beam. The extraordinary incident light beam is spatially modulated by the liquid crystal layer 8 as it passes through towards the quarter wave retardation layer 41. The ordinary incident light beam and the extraordinary incident light beam undergo one quarter wave relative phase delay by passing, in the incident direction, through the quarter wave retardation layer 41. Both the ordinary incident light beam and the extraordinary incident light beam are reflected by reflective layer 9 into a corresponding ordinary reflected light beam and an extraordinary reflected light beam. As the ordinary reflected light beam and the extraordinary reflected light beam pass a second time through the quarter wave retardation layer 41, both reflected beams undergo a second quarter wave relative phase delay. The induced full half wave relative phase delay induced results in each reflected beam being polarized in the other polarization plane (perpendicular) with respect to the corresponding incident polarization plane. As the reflected, originally ordinary polarized beam passes the second time (as an extraordinary polarized beam) through the liquid crystal layer 8, it is spatially modulated by the liquid crystal layer 8, while the reflected, originally extraordinary polarized, beam passes (as an ordinary polarized beam) the second time through the liquid crystal layer 8 unaffected. Both spatially modulated reflected beams make up the spatially modulated reflected light beam 4. More generically layer 41 is a polarization rotator which exchanges the polarization of the ordinary and extraordinary beams (with respect to the first splitting of the unpolarized incident beam by the first passage of the unpolarized beam through the LC layer 8) between the first and second passage of the beams through the LC layer 8.

In accordance with an implementation, the use of a single LC layer 8 and a birefringence plate 41 within the optical reflective device 11 provides for polarization independent operation, according to a non-limiting implementation of the proposed solution. Birefringence plate 41 has an axis oriented at angle α with respect to the director of the LC layer 8. It has the role of providing a modified polarization (compared to the first passage, e.g. being rotated at 90°) during the second passage of the light through the same LC layer; which makes the overall device 11 operation polarization independent.

FIGS. 8A and 8B schematically illustrate an important alternative of above mentioned LC mirrors 11 providing tunable phase curvature, in which there is no electrode (12) material in the optical path of light (localized close to the z axis), according to a non-limiting implementation of the proposed solution. There is shown a tunable LC mirror 11 with high transparency and high optical power resistance. In this configuration, the incident light beam 2 and the reflected light beam 4 do not traverse any electrode (12) layer material as the clear optical aperture of the device 11 is as large as a core of an annular electrode 12. In a variant, the birefringent plate 41 can be replaced by a cross-oriented LC layer 81, a quarter wave retarding layer or can be completely removed (resulting in a polarization dependent LC mirror 11 when acceptable or desired). The back electrode 10 can be chosen to have different forms, including those (100) shown in FIGS. 5A to 5G. The front electrode 12 can be formed of one, two or more segments for tilt and angular control; an example of two annular segments 121 and 122 being illustrated in FIG. 8B. Coupled to a controller, the two annular segments 121 and 122 can be configured not only to focus the reflected light beam 4 but also to steer the reflected light beam 4. FIG. 8C illustrates, in plan view, a front electrode 12 segmented into quarters and an optical steering and image stabilization controller 110 configured to operate the tunable mirror 11 not only to control the focus of, and to steer, the reflected light beam 4, but also to correct for aberrations such as coma, astigmatism, etc. While four segments are illustrated, the invention is not limited thereto, six, eight or more segments can be employed to provide optical image stabilization and aberration control. An appropriate optical image stabilization controller 110 responds to image characteristics of the optical field passing through the LC mirror 11 and provides instructions to corresponding signal drivers (not shown) for each segment. Further description is provided in US Patent Application US 2012/0257131 claiming priority from U.S. Provisional Patent Application Ser. No. 61/289,995 filed Dec. 23, 2009, the entireties of which are incorporated herein by reference.

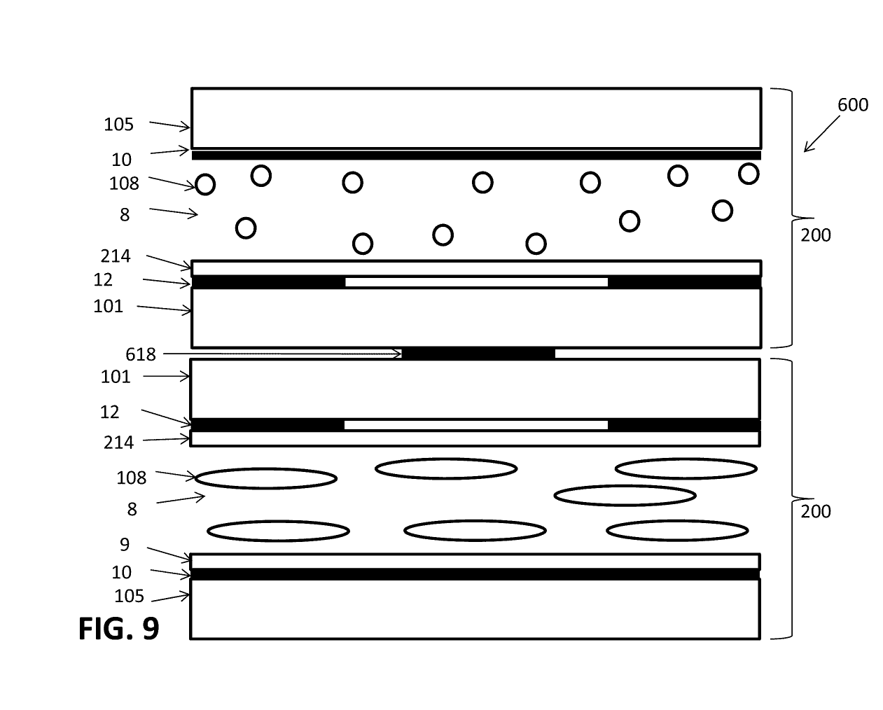

To resolve aberration (wavefront) problems, PCT international publication WO2012/079178, which is incorporated herein by reference, has introduced a geometry 600, where a transparent floating (non-connected) conductive layer (generally in the form of a disc) 618 is introduced between the two cross oriented LC layers 8 of a pair of half LC lenses 200 (each polarization dependent in operation), used in a polarization independent tunable mirror geometry 600, illustrated in FIG. 9. The presence of the floating conductive layer 618 improves significantly (compared to prior art designs) the wavefront profile and the Modulation Transfer Function (MTF) of devices using such LC mirror 600. In addition, a unique control signal required for driving the tunable mirror 600 is very low (power/signal amplitude) and the device 600 operates essentially by frequency control. In this geometry, a Weakly Conductive Layer (WCL) layer 214 can be employed close to the LC layers 8, and corresponding Hole Patterned Electrodes (HPE) 12. Layers 101 and 105 correspond to substrates on which the geometry is manufactured. Layers 101 can be dielectric. The top substrate 105 is transparent while the bottom substrate 105 may be behind the reflective surface 9 (not necessarily transparent). The top “back” electrode 10 is transparent for example made of Indium Tin Oxide (ITO) while the bottom “back” electrode 10 may be metallic, for example highly reflective Al, Au, etc. depending on the (optical frequency) band of operation of the device 11 (600). If the bottom back electrode 10 is highly reflective, then the reflective layer 9 itself can be omitted. LC molecular directors 108 of each LC layer 8 are illustrated cross oriented with respect to each other.

An alternative approach was proposed to resolve poor WCL 214 manufacturing repeatability and undesired wavefront aberrations by N. Hashimoto, “Liquid Crystal Optical Element and Method for Manufacturing Thereof”, US patent, U.S. Pat. No. 7,619,713 B2, Nov. 17, 2009, and is shown in FIG. 10A implemented in a tunable mirror geometry 700 in accordance with the proposed solution. The basic difference in this geometry 700, compared to the geometry 600 illustrated in FIG. 9, is the absence of the WCL 214. In fact, Hashimoto proposes the use of multiple optically transparent Concentric Ring Shaped Electrodes (CRSE) 702 interconnected via high resistivity “bridges” 720 (the schematic side view is shown in FIG. 10A and the top view is shown in FIG. 10B). This “resistively-bridged” structure plays a similar role as the WCL (214) in creating a (voltage) potential spatial profile over the aperture. The advantage of this approach is that the individual resistivity values (R1, R2, etc.) of the bridges 720 can be adjusted to obtain a desired wavefront. Also, two small voltages V1 (206) and V2 (706) are needed, applied to the center 712 and to the periphery of the external ring shaped electrode 12 (respectively) with the electrode 10 being grounded to drive the tunable mirror 700. Again the “bottom back” electrode 10 may be metallic, for example highly reflective Al, Au, etc. depending on the (optical frequency) band of operation of the device 11 (700). If the bottom back electrode 10 is highly reflective, then the reflective layer 9 itself can be omitted. FIG. 10C is a plan view of a non-limiting segmented electrode structure 12 for use in controlling aberrations for a LC tunable mirror 11 geometry 700 as illustrated in FIGS. 10A and 10B wherein CRSE 1712 are segmented (less or more than four subsegments per ring 702 can be used with due change in control.)

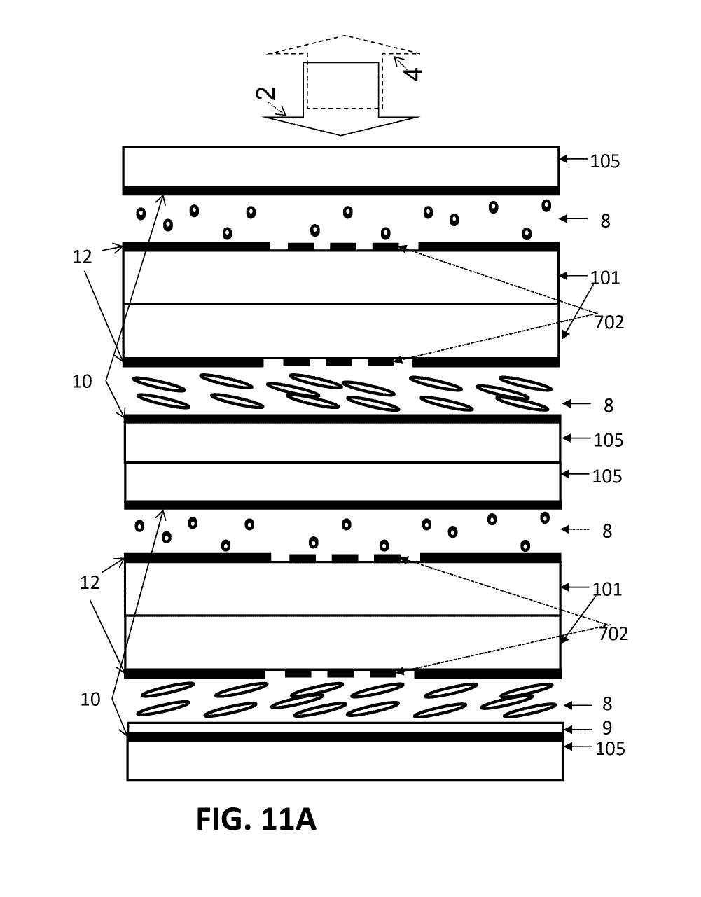

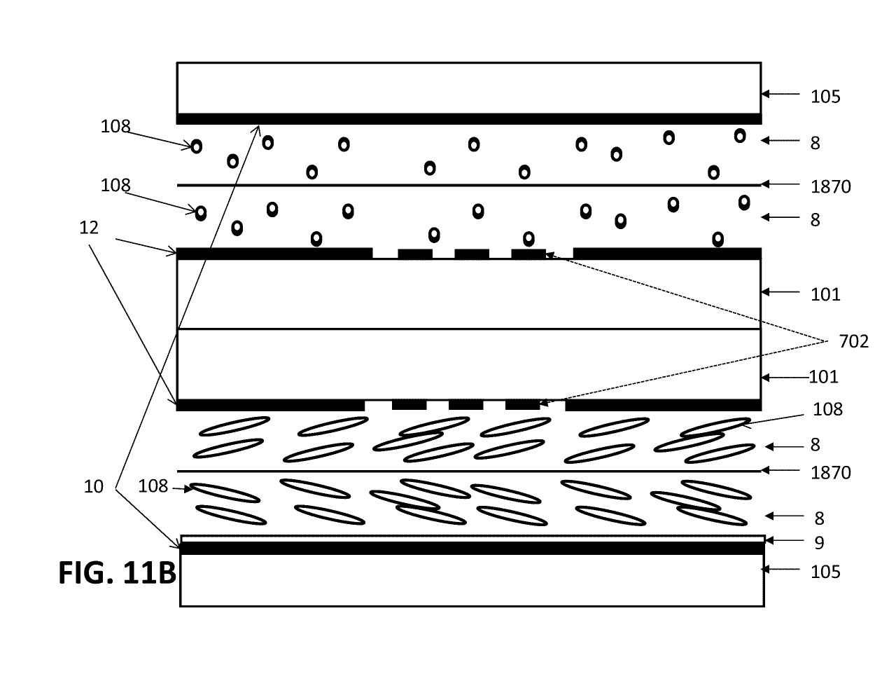

In accordance with another embodiment of the proposed solution FIG. 11A illustrates a dual LC lens polarization independent tunable LC mirror 11 structure employing two polarization independent LC lenses, without limiting the invention, for example each having the layer geometry 700 illustrated in FIG. 10A herein wherein corresponding LC layers 8 in each polarization independent LC lens have directors (108) oriented in opposing directions. Besides providing double the optical power of each polarization independent LC lens, the overall geometry also provides a reduction in image splitting between the two polarizations of light as described in US published Patent Application 2011/0090415 claiming priority from U.S. Provisional Patent Application 61/074,651 filed 2008 Jun. 6, the entireties of which are incorporated herein by reference. While the tunable LC mirror 11 geometry illustrated in FIG. 11A includes dual LC lenses doubling the thickness of the layered geometry, a reduction in the overall layered geometry is possible as illustrated in FIG. 11B. The polarization independent LC mirror 11 layered geometry illustrated in FIG. 11B employs the same electrode structure as illustrated, for example, in FIG. 10A to drive dual adjacent LC layers 8 with LC directors (108) oriented in opposing directions. A rubbed or stretched membrane 1870 is employed as an alignment layer between adjacent LC layers 8. In accordance with yet another embodiment of the proposed solution, the reduction in image splitting can also be achieved by shifting each polarization dependent LC lens in a polarization independent LC lens geometry, for example, as illustrated in FIG. 10A to counteract image shifts between the two polarizations as described in PCT international publication WO 2014/138974 filed 2014 Mar. 12 claiming priority from U.S. Provisional Patent Application 61/800,620 filed 2013 Mar. 15, which are incorporated herein by reference.

Without limiting the invention, in the above described embodiments the variability of the optical power of the LC tunable mirror 11 is unipolar, i.e. either negative optical power or positive optical power.

In accordance with another embodiment of the proposed solution, the optical power tuning range LC tunable mirror 1100 employing CRSEs 702 can almost be doubled by employing and splitting a top Uniform Control Electrode (UCE) into a Hole Patterned Electrode (HPE2) 1732 and a Control Disc Electrode (CDE) 1734 which are manufactured on the same substrate (101) surface, as schematically illustrated in FIGS. 12A and 12B, or HPE2 1732 and UCE 1736 manufactured on different substrate surfaces, as schematically illustrated in FIG. 13. A transparent spacing layer 1007 is employed for separation. Further description is provided in U.S. patent application Ser. No. 13/371,352 claiming priority from U.S. Provisional Patent Application 61/441,647 filed 2011 Feb. 10 and in International Patent Application WO 2014/071530 filed 12 Nov. 2013 claiming priority from U.S. Provisional Patent Application 61/725,021 filed 2012 Nov. 11, the entireties of which are incorporated herein by reference. These geometries provide bipolar functionality of the LC tunable mirror 1100, from negative to positive optical power and vice versa. The diameter of the HPE2 1732 is smaller than that electrode “HPE1” 12. The driving method includes:

For positive OP tuning the V_CDE=V_HPE2 is smaller than the V_HPE1; and

For negative OP tuning the V_CDE is larger than the V_HPE1, and V_HPE2 is kept either floating or with biased voltage V_HPE1≦V_HPE2≦V_CDE.

It should be noted that, in the mirror configurations shown in the above embodiments, reflector 9 can be removed and the bottom back electrode 10 itself can play the role of the reflector 9. In the case of using a non-uniform electrode (such as the elements 10, 15, 16, 18 of FIGS. 5A to 5G, etc.) as the back electrode 10, the electrical function of the electrode and the optical function of reflection should be attuned (consistent). The addition of dielectric or semiconductor materials, or their combination can, at least partially, decouple those functions making its implementation and use easier. For example, the combination of both functions, light reflection and creation of non-uniform excitation, can be decoupled by using, without limiting the invention thereto, a concave metallic structure which can create a non-uniform electric field (13), as well some dielectric layers can be deposited on the concave metal electrode to perform the reflection.

It should be noted that, in the above configurations, the LC material (and its electro-optic excitation) can be replaced by a combined liquid or polymer composite (along with a thermal, acoustic or mechanical excitation) and still provide the same mirror performance.

It should be noted also that, in the above configurations, the excitation modes (mechanisms) used can be different, such as electric, magnetic, thermal, piezo, acoustic, etc.

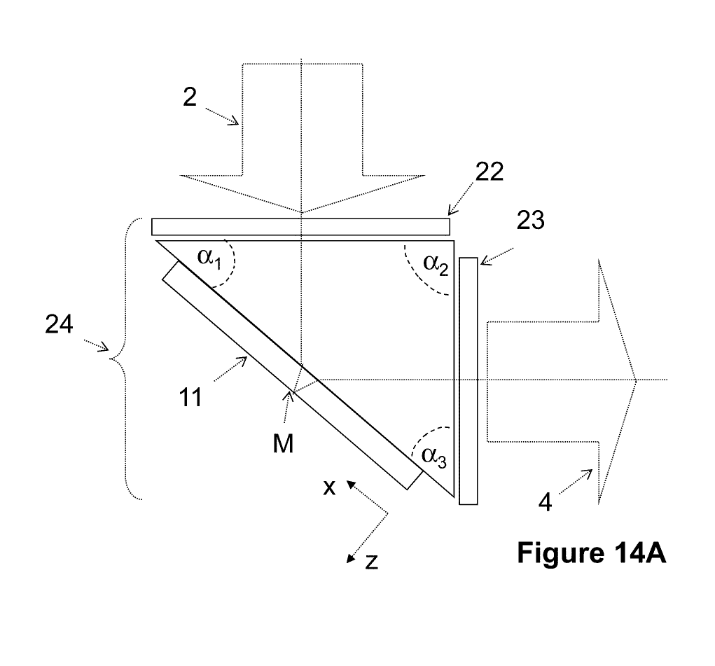

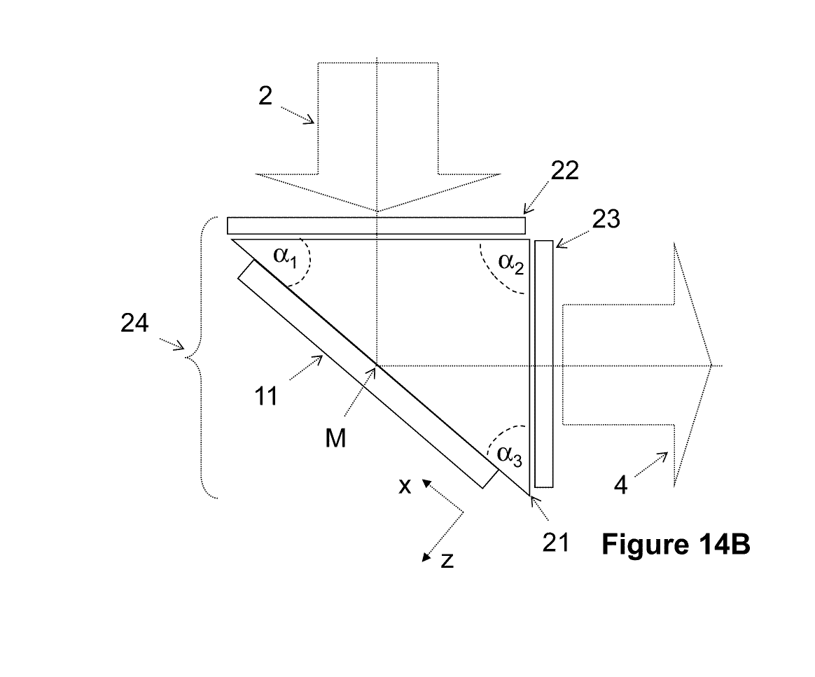

FIG. 14A illustrates a geometry of (configuration for) an angle reflecting tunable mirror 11, according to a non-limiting implementation of the proposed solution. More specifically, there is illustrated a tilted incidence optical reflective device 24 with tunable phase curvature, including a prism-like body 21 made with appropriate geometrical parameters α₁, α₂ and α₃ and of an appropriate optical material (for example index of refraction); a pair of conditioning optical elements 22, 23; and a tunable mirror structure 11 adjacent to the back surface of the prism-like structure 21. The main reflecting surface M is behind the “back surface” of dynamically controllable material (8) of the tunable mirror 11, allowing a double passage of reflected light through that material (8). In a specific example, the back surface M of the tunable element 11 (exposed to air or other lower refractive index element/media) can be used as a reflective surface (9) via functionality (by mechanisms) described herein or via total internal reflection, removing the need for a fixed mirror (9) on the back of the element 11.

In a variant example, the surface of the prism 21 itself (in the interface between the prism and the controllable material (8) of the tunable mirror 11, such as LC or composite polymer, etc.) could provide the total internal reflection, as shown in FIG. 14B. In this case, the non-uniform excitation of the controllable material (8) (e.g., LC or polymer layer, e.g. 0,) placed in the immediate vicinity of the prism 21 surface can be used to generate electrically controllable non-uniform total internal reflection. The reflecting surface M is essentially the “entrance” surface of dynamically controllable material (8) allowing an evanescent penetration of the reflected light into that material (8). This and previous arrangements can provide the spatially non-uniform and dynamically controllable phase and amplitude modulation of the reflected light.

In a particular embodiment, where the gradient of excitation and the respective gradient of refractive index are, for example, circular, the reflected (from the above mentioned structures) light intensity can be modulated in a radial direction providing a dynamically tunable aperture function in reflection geometry. This is because the total internal reflection depends upon the difference of refractive indices of both sides of the interface M. The non-refractive index on one side provides non-uniform amplitude (intensity) of reflection.

Note that in the case of both configurations shown in FIGS. 14A and 14B, the liquid crystal layer (8) can always be chosen with uniform, planar, tilted, hybrid or other configurations of the LC director (108) distribution.

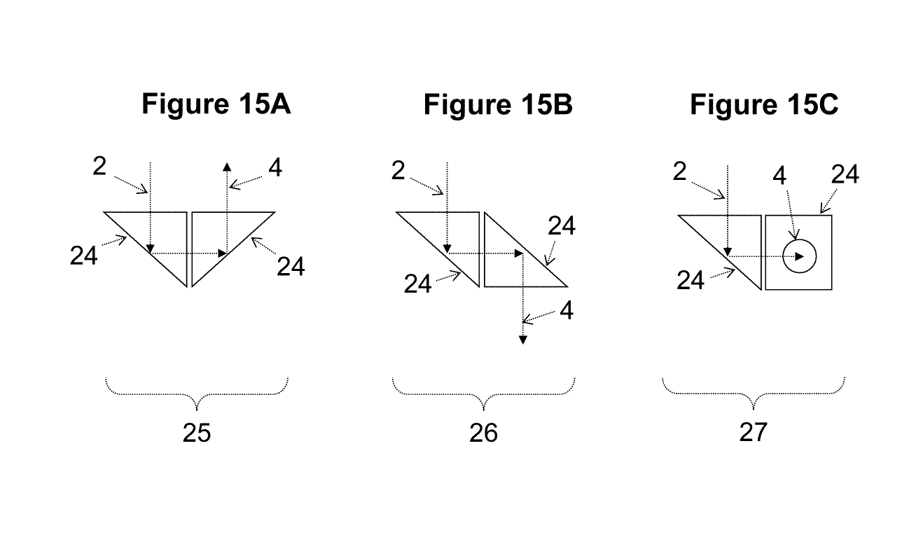

FIGS. 15A to 15C illustrate different non-limiting examples of the arrangement of tilted incidence tunable reflective devices 24, 241 having FIG. 15A counter propagating 25, FIG. 15B co-propagating 26 and FIG. 15C cross-propagating 27 incident 2 and reflected 4 light beams.

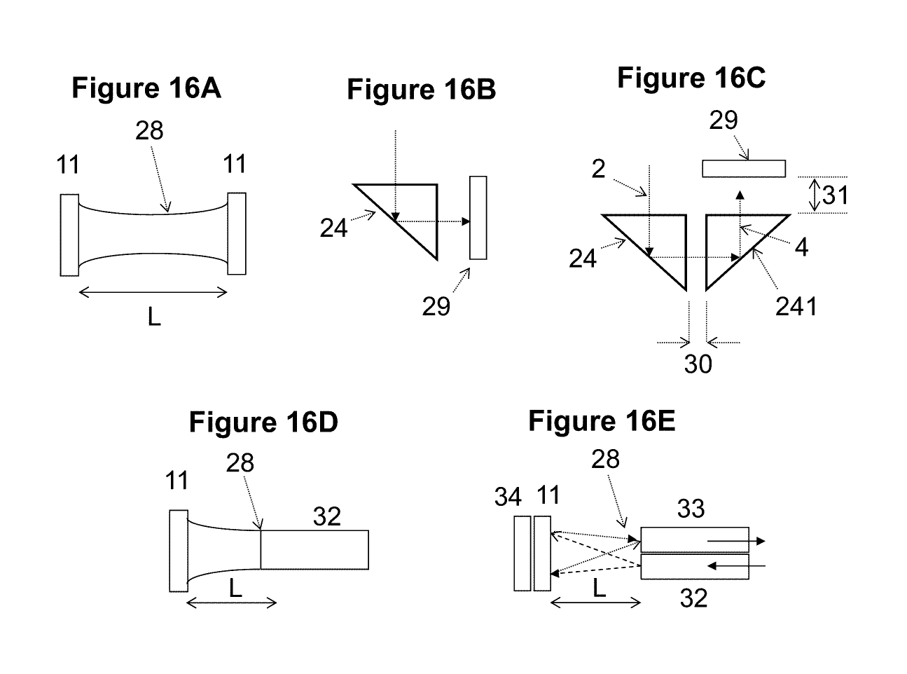

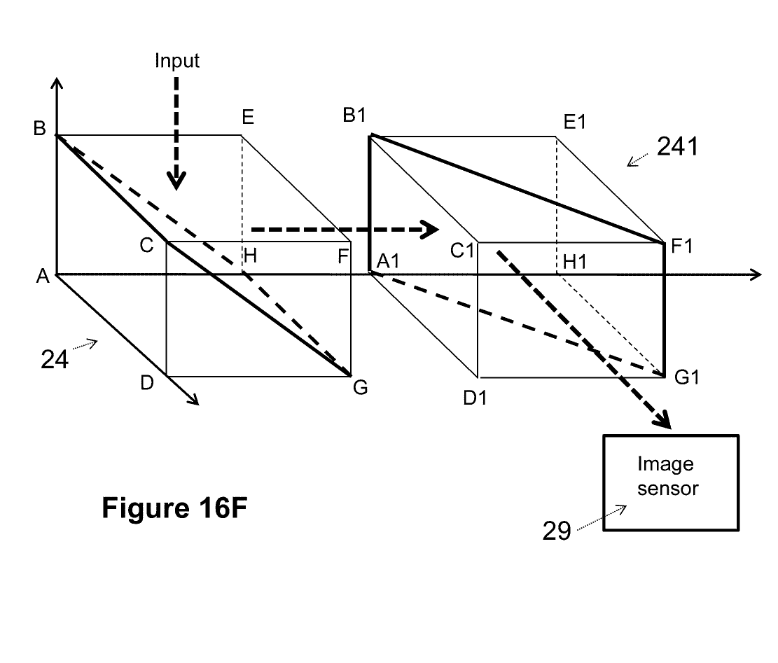

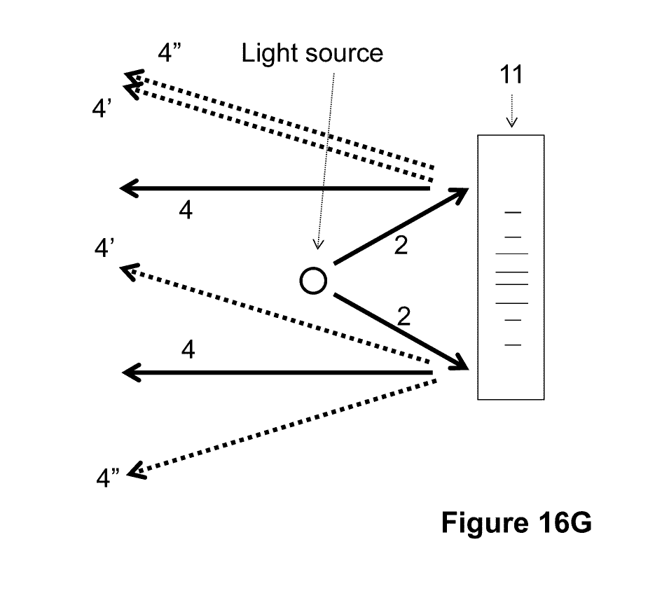

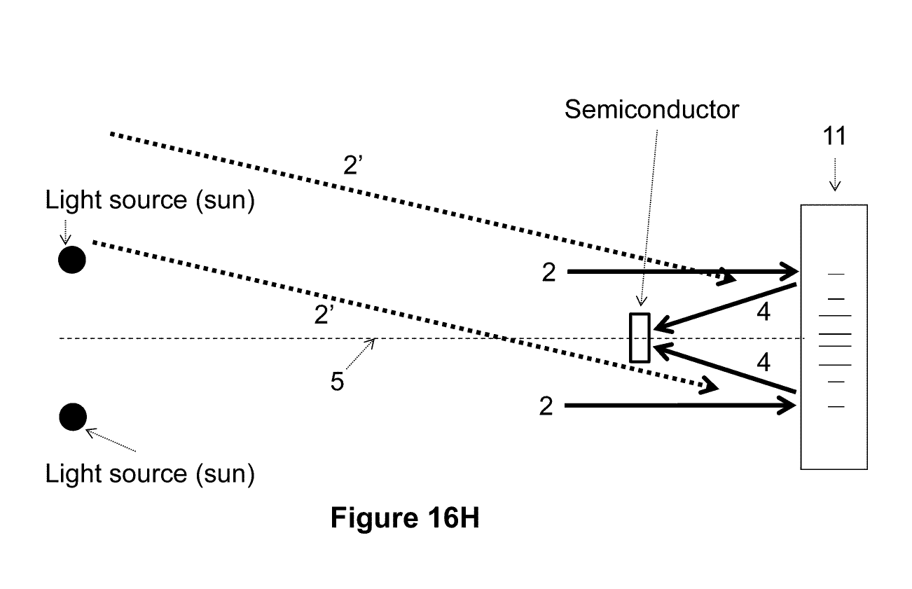

FIGS. 16A to 16E illustrate various non-limiting examples of application of a tunable reflective device (11, 24) as described above. For example, such a tunable reflective device (11, 24) can be used to build photonic (optical) devices. For example, a tunable laser resonator for shaping a light beam profile 28 is schematically illustrated in FIG. 16A. The reflective device 11 is used to tune the radial distribution of the curvature and/or of the intensity of the reflected light. Imaging systems are another example wherein a tunable self-focusing (24) imaging system with image sensor or observation plane 29 is schematically illustrated in FIG. 16B. An optical zoom system composed with tunable reflectors 24 & 241 (for example generating cross propagation directions) and an image sensor or observation plane 29 positioned at appropriate distances, 30 and 31, is schematically illustrated in FIG. 16C. A waveguide or fiber laser 32 is schematically illustrated in FIG. 16D. And, a variable optical attenuator for controlling light coming from an input fiber 32 to be reflected into an output fiber 33 is schematically illustrated in FIG. 16E. (typical light divergence from SMF28 is ±6°) An optional photodetector 34 can be added in the back of a partially reflecting tunable mirror 11.

FIG. 16F illustrates a perspective view of an optical system providing both optical zoom and image stabilization functionalities. A “motion-less” optical zoom and image stabilization device can be built by using two tunable mirrors 24, 241 integrated on reflecting surfaces BHGC and A1B1F1G1, each tunable mirror on the corresponding reflection arm having a control electrode (12) pattern as illustrated in FIG. 10C. The input plane is BEFC. The image sensor 29 may be integrated on the surface D1C1F1G1. The tunable mirrors 24, 241 must be spaced and oriented in a predetermined way and they (at least one of them) must use transversally segmented electrodes (151, 152) to steer light. This can be an easy-to-assemble motion-less compact optical zoom and image stabilization device. Optionally, a tunable liquid crystal lens can be added on the BEFC or EFGH surfaces.

FIG. 16G illustrates a cross-sectional view through a beam steering optical device. A tunable mirror 11 can be used to steer light from a source, such as a Light Emitting Diode or Laser. Various modes of operation are possible. The original incident light rays (2) can be reflected in a normal collimated way (4), tilted/steered collimated way (4′) or with decreased or increased divergence 4″, and other options.

FIG. 16H illustrates a cross-sectional view through a light source tracking device, for example an angular tracking device for solar concentrators. Tunable mirror 11 can be used to optimize the operation and cost of photovoltaic solar concentrators combining reflective focusing and steering functions.

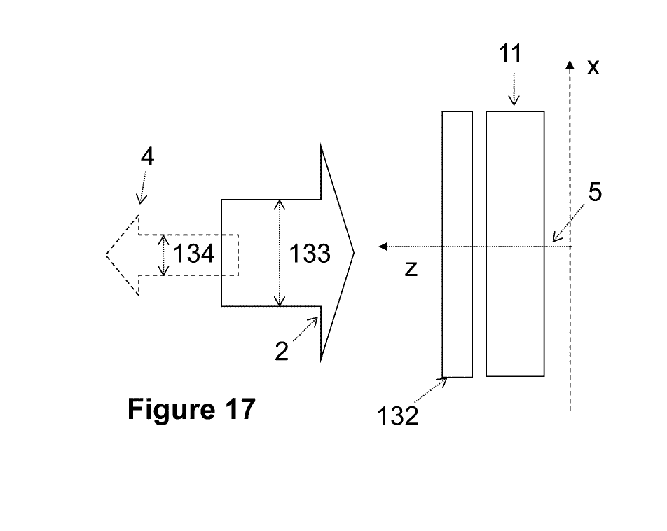

FIG. 17 illustrates an example of a use of tunable reflective elements to build a dynamically variable reflective pinhole or diaphragm. More specifically, there is illustrated a dynamically variable and spatially non-uniform reflectivity mirror using a polarization dependent tunable mirror 11 and a polarization-sensitive optical element 132, such as anisotropically absorbing, scattering, refracting or reflecting material or element, polarization beam splitter, tilted or angle polished interfaces (for example, a glass plate or an interface of an active medium, etc.). The optical axis 5 of mirror 11 makes a predetermined angle with respect to the anisotropy axis z of the supporting polarization-sensitive material 132. The diameter 134 of the reflected beam 4 can be controllably reduced with respect to the diameter 133 of the incident beam 2. Both phase curvature and amplitude/diameter are affected.

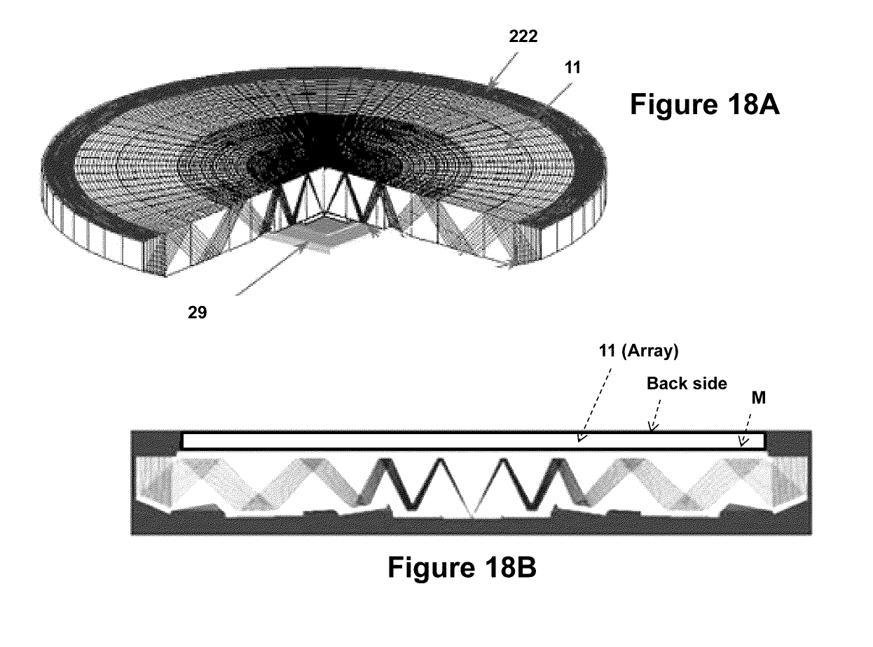

FIGS. 18A and 18B illustrate an example of the use of tunable reflective elements 11 to build for example, in combination with an Origami-like lens/camera an electrically variable flat imaging telephoto lens system with optical auto-focus and/or zoom function. As illustrated in FIG. 18B an array of tunable mirrors 11 can form the “back” side of the Origami lens. While FIG. 18A illustrates such an Origami lens for imaging applications, the invention is not limited thereto. The same Origami lens illustrated in FIGS. 18A and 18B can form part of an intraocular prosthesis configured to replace the natural lens of an eye to provide augmented vision (for example, to vision impaired individuals). For certainty, the annular peripheral area 222 (input aperture) is greater than a central pupillary area of the eye thus gathering, and delivering to the retina, an increased light flux. While such an implant (prosthesis) would deliver increased light flux in all conditions, the invention is not limited thereto:

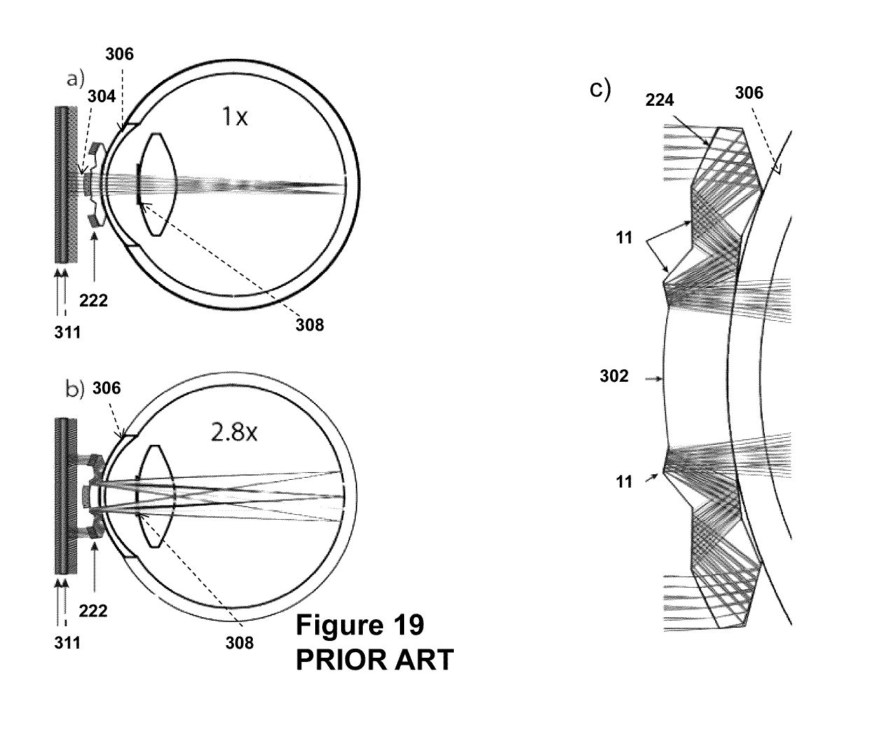

FIGS. 19a) to 19c) schematically illustrate a contact lens prosthesis 224 for a eye configured to selectively provide low light augmented vision and/or telescopic function as described by E. Tremblay et al., in “Switchable Telescopic Contact Lens” Optics Express, Vol. 21, Issue 13, pp. 15980-15986, 2013. A central opening 302 is provided to allow passage of incident light 304 as it normally would through the cornea 306 and through the eye pupil 308. It is noted that what is illustrated and described by Tremblay does not represent a working integrated solution as an additional external switching element 311 (for example like in 3D cinema) is required to make the switch between FIG. 19a) and FIG. 19b) operation.

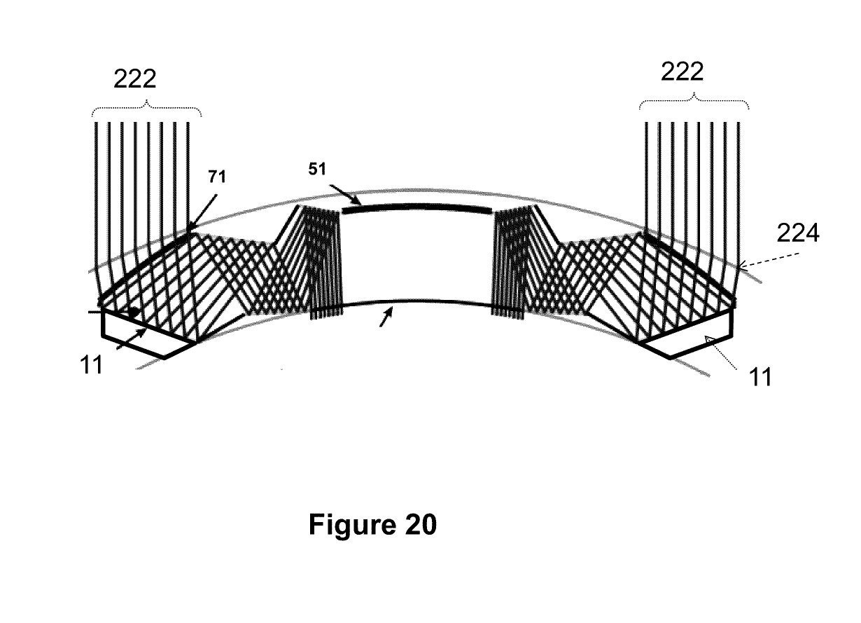

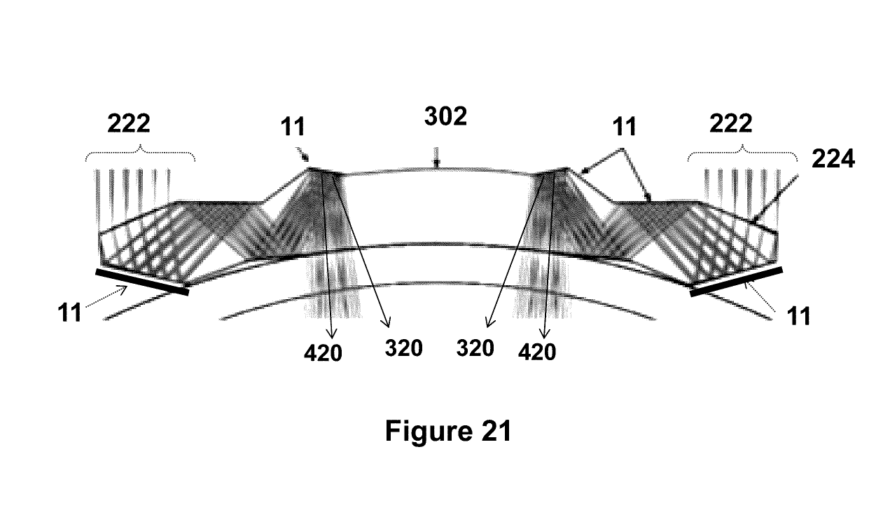

In accordance with the proposed solution, FIG. 20 illustrates an integrated solution with the addition of an annular tunable mirror 11 and integrated polarizers 51, 71 for coordinating the operation of the device 224 with the operation of the pupil. The pupil 308 has a small diameter during ample light flux (daylight) conditions and the annular tunable mirror 11 can be employed to divert light incident on the peripheral annular ring 222 onto the same retinal area as light 304 incident on the pupil 308 in order to increase light flux (for visually impaired individuals) in order to enhance central vision. As illustrated in FIG. 21, the same geometry can also be employed to provide vision with light incident from the periphery 222 of the structure redirecting incident light towards usable portions of the retina and away from damaged portions of the retina. The arrangements illustrated in both FIGS. 20 and 21 can also employ the (variable) tunable annular mirror 11 to automatically focus the incident light 222 onto the retina. In accordance with another implementation of the embodiment of the proposed solution, the use of segmented electrodes (for example see FIG. 8B, FIG. 8C and FIG. 10C) in annular tunable mirror 11 enables angular steering for example for redirecting the incident light 222 towards usable retinal regions for example away from retinal scars.

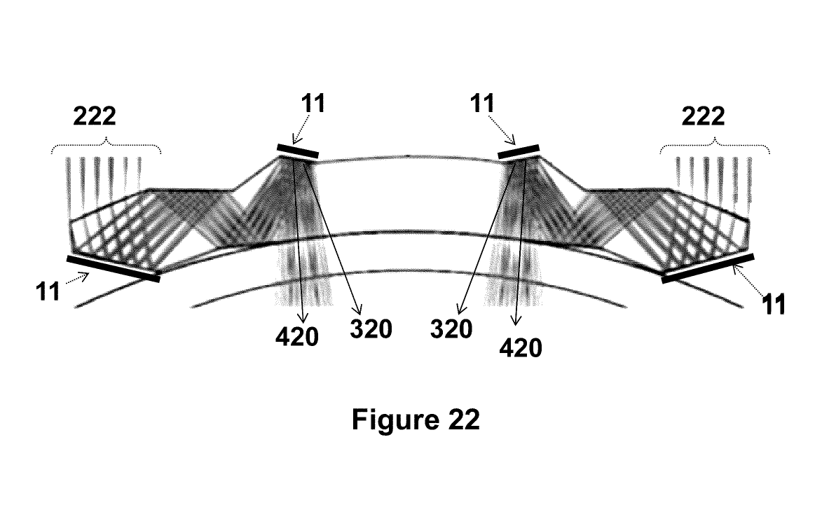

The implementation of the proposed solution illustrated in FIG. 22 employs a second annular tunable mirror 11 in a contact lens 224, combination of tunable mirrors which can also provide telescopic zoom function in addition to the above mentioned functionality.

While in implementations illustrated in FIGS. 20 to 22 light incident on the periphery 222 of the device 224 is redirected towards the retina at the same time as light 304 incident on the center of the device (224), in accordance with another implementation of the proposed solution, the addition of polarizers (51, 71) and a switchable polarization rotator (81) (such as a twisted nematic liquid crystal cell) on all optical paths can provide for separate control between central 320 and peripheral 420 ray (donut) vision. For example, orthogonally oriented polarizers 51 and 71 are employed on the incident side, while polarizer 61 is oriented in the same polarization direction as either polarizer 51 or polarizer 71. A switchable polarization rotator 81 configured to pass light through unaffected in an inactive state (without polarization rotation) and to induce a 90° polarization rotation in an active state can be used in front of polarizer 61. For example, if polarizers 51 and 61 have the same polarization orientation, the peripheral 420 rays are cut out if the polarization rotator 81 is inactive and the wearer sees the central rays only. When the polarization rotator 81 is active, the rotation of polarization by 90° will cut the central 320 rays allowing only the transmission of the peripheral 420 rays.

For example the polarization rotator 81 can be activated during dim light conditions, when the pupil 308 is enlarged, to gather, and to redirect towards the retina, light incident on a peripheral annular region 222 of the (intraocular/contact) lens 224 having a greater area thus providing augmented vision.

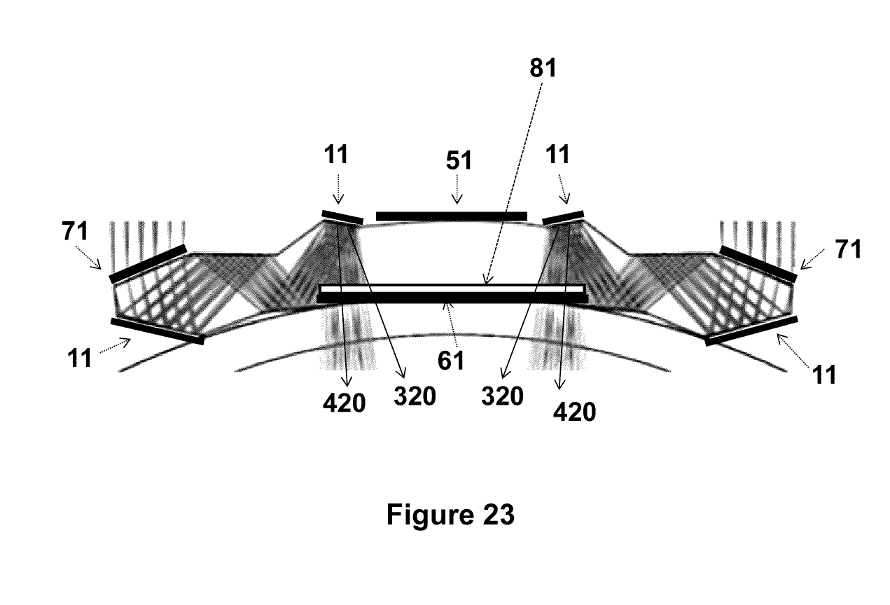

For certainty, the geometries illustrated in FIGS. 20 to 23 can also be implemented in a standard gas permeable contact lens 224 in the form of local disks and/or discrete ring structure(s) to enable some gas diffusion therethrough for contact lens (224) applications.

For certainty, the geometries illustrated in FIGS. 20 to 23 can also be implemented in an intraocular prosthesis replacing or augmenting the natural lens of the eye. (Not shown are electrical power sources and triggering (control) system(s).)

It may be appreciated that providing such integrated and independently operational tunable contact lenses 224 and intraocular lens implants (from many discrete elements) enables customer-side adaptation therefore relaxing manufacturing tolerances.

It may be appreciated that various material compositions, various controllable material (e.g., LC, polymer, liquid, composite, etc.) layers, various electrodes, various director alignments, various geometrical forms, etc. can be used to fabricate the same device, which may provide “hidden” state for optical waves and very strong dielectric permittivity contrast for low frequency electric fields.

It is important to note that the while above-described embodiments of the proposed solution have been presented for illustration purposes, additional variants and modifications are possible and should not be excluded from the scope of the claims.

It should also be appreciated by the reader that various optical devices can be developed using one or more combinations of devices described above.

What is claimed is:

1. A variable optical device for controlling properties of reflected light, the device comprising:
a light reflecting structure;
a layer of continuous non-pixelated dynamically controllable material including one of a liquid crystal mixture and a polymer composite; and
an excitation source for generating an excitation field acting on said layer of dynamically controllable material having a variable index of refraction sensitive to said excitation field,
wherein an electrical drive signal applied to said excitation source causes a change of optical properties in said layer of dynamically controllable material to provide a spatially varying change in light reflection having at least one of a desired phase curvature and a desired amplitude modulation profile.

2. A device as defined in claim 1, wherein said layer of dynamically controllable material is planar.

3. A device as defined in claim 1 or 2, wherein said layer of dynamically controllable material has a spatially variable index of refraction.

4. A device as defined in claim 3, wherein said layer of dynamically controllable material comprises nematic liquid crystal material interspersed into a spatially nonuniform polymer stabilized matrix.

5. A device as defined in claim 1 or 2, wherein said layer of dynamically controllable material comprises nematic liquid crystal material sandwiched between a pair of alignment layers.

6. A device as defined in claim 5, wherein each of said pair of alignment layers has an alignment direction, said pair of alignment layers being oriented in one of same direction and opposing direction with respect to each other.

7. A device as defined in any of claims 1 to 6, wherein said excitation source includes an electrode system arranged to generate said excitation field.

8. A device as defined in claim 7, wherein said electrode system includes first and second groups of electrodes arranged on opposite sides of said layer of dynamically controllable material.

9. A device as defined in claim 7 or 8, wherein said excitation field generated by said electrode system is spatially non-uniform.

10. A device as defined in claim 9, wherein said spatial non-uniform electrode system is configured to generate a spatially non-uniform field obtained by lateral attenuation of a potential across a combination of electrode system geometry and by electrical and optical properties of adjacent materials without using individual control of a plurality of pixels.

11. A device as defined in any of claims 7 to 10, wherein said electrical drive signal is time variant for modulating said excitation field, said electrical signal having one of a time variant amplitude and a time variant frequency.

12. A device as defined in any of claims 8 to 9, wherein said first group of electrodes is at least one of non-uniform and segmented, and said second electrode group is uniform.

13. A device as defined in claim 12, wherein said first group of non-uniform electrodes includes a hole patterned electrode and a weakly electrically conductive layer.

14. A device as defined in any one of claims 7 to 13, wherein said excitation field is one of: an electric field, a magnetic field and a thermal excitation.

15. A device as defined in any one of claims 1 to 6, wherein said excitation field includes an acoustic excitation.

16. A device as defined in any one of claims 5 to 14, wherein said layer of liquid crystal mixture is characterized by one of: a spatially non-uniform liquid crystal cell alignment and a spatially uniform liquid crystal cell alignment.

17. A device as defined in any one of claims 1 to 16, wherein said light reflecting structure is one of a metal mirror, a dielectric mirror, a plurality of dielectric layers and a total internal reflection interface.

18. A tunable optical device for controlling the properties of reflected light, said device having a variable light reflection phase curvature, controlled essentially by an electrical drive signal.

19. A tunable optical device as defined in claim 19, further comprising an active polarization rotator configured to select between two polarizations of light.

20. A tunable optical device for controlling the properties of reflected light, said device having a variable light reflection amplitude spatial distribution, controlled essentially by an electrical drive signal.

21. A tunable optical device as defined in any one of claims 1 to 20, in combination with additional optics to form incident and reflected beams in counter propagation, co-propagation and angled (e.g. cross) propagation geometries.

22. A combination of at least two controllable non-uniformly reflective devices as claimed in any of claims 1 to 21 and additional optics including an image sensor to form one of an optical zoom system, an autofocus system and image stabilization system in a mobile camera.

23. An array of controllable non-uniformly reflective devices as claimed in any of claims 1 to 21 in combination with additional optics, such as an Origami-kind lens, to form one of an optical zoom system and an autofocus system, wherein said array is one of: periodic, aperiodic, concentric and linear.

24. A contact lens or an intraocular lens for enhancing vision, the lens comprising:
an array of controllable non-uniformly reflective devices in combination with additional optics, such as an Origami-kind lens as defined in claim 23;
a first integrated polarizer layer having a first polarizing orientation over a central area of said Origami lens;
a second integrated polarizer layer having a second polarizing orientation over a peripheral area of said Origami lens; and
an integrated polarization rotator layer in a combined optical path of incident light passing through said first and second polarizer, said polarization rotator being configured to select between central area vision and peripheral vision for selecting between normal and zoomed vision.

25. A lens as defined in claim 24, at least one of said non-uniform reflective devices comprising a group of segmented electrodes in a transversal plane configured to steer reflected light inside an eye to change an imaging area on a retina of said eye.

26. A device as defined in any one of claims 7 to 25, wherein the excitation source comprises a pair of electrodes, one of which is reflective and provides said light reflecting surface.

27. A device as defined in any one of claims 1 to 26, wherein a desired spatial distribution of amplitude of reflected light is also controlled.

28. A device as defined in any one of claims 1 to 27, wherein one of a phase and an amplitude of reflected light is controlled using two liquid crystal material layers having director distributions arranged in cross oriented directions so as to provide polarization independent operation.

29. A device as defined in any one of claims 1 to 27, wherein a phase or amplitude of reflected light is controlled using liquid crystal material arranged in a single layer in combination with a birefringent plate so as to provide polarization independent operation.

30. An array of controllable non-uniformly reflective devices as claimed in any of claims 1 to 21 in combination with at least one photovoltaic cell configured to steer solar incident light to compensate for solar movement.

31. An array as defined in claim 30, wherein said array is further configured to focus said solar incident light onto said photovoltaic cell.
